(19) United States
(12) Patent Application Publication
(10) Pub. No.: US 2009/0199858 A1
(43) Pub. Date: Aug. 13, 2009

(54) Cannula cover

(76) Inventors: Robert Carl Hagberg, Butler, MO (US); Bruce Alan Wachter, Granby, MO (US)

Correspondence Address: Robert Carl Hagberg, 508 West Ave., Butler, MO, 64730, US

(21) Appl. No.: 12/321,850

(22) Filed: Jan. 27, 2009

Related U.S. Application Data
(60) Provisional application No. 61/065,326, filed on Feb. 9, 2008.

(51) Int. Cl. A61M 15/08

(52) U.S. Cl. 128/207.18

(57) ABSTRACT

The Cannula Cover is an integral simple, movable, sanitary field enclosure. Conceived as an addition to the current Nasal O2 Cannulas on the market. It address's the unresolved documented upper respiratory nosocomial infection potentials, related to the unsanitary practices observed in both clinical and private use situations, thus resolving a problem of more than 3 decades, which has been directly related to unprotected Nasal O2 Cannulas.

### CROSS-REFERENCE TO RELATED APPLICATIONS

| JP 2001000550 | | |
|---|---|---|
| WO 2007/11935 | | |
| 7,127,278 | Melker, et al. | Oct. 24, 2006 |
| 4,648,398 | Agdanowski | Mar. 10, 1987 |
| 00/718,785 | McNary | Jan. 20, 1903 |
| 2,735,432 | Hudson | February 1956 |
| 2,868,199 | Hudson | January 1959 |
| 2,931,358 | Sheridan | April 1960 |
| 3,400,714 | Sheridan | September 1968 |
| 3,726,275 | Jackson | April 1973 |
| 4,156,426 | Gold | May 1979 |
| 4,367,735 | Dali | January 1983 |
| D309,960 | Applebaugh | Aug. 14, 1990 |
| 4,106,505 | Salter | Aug. 15, 1978 |
| 4,685,456 | Smart | August 1987 |
| 4,774,946 | Ackerman | October 1988 |
| 4,818,320 | Wetchselbaum | April 1989 |
| 2,763,263 | Ellman | September 1956 |
| 3,754,552 | King | August 1973 |
| 1,056,255 | Cadman | Mar. 18, 1913 |
| 2,693,800 | Caldwell | November 1954 |
| 6,679,265 | Strickland, et al. | Jan. 20, 2004 |
| 3,463,309 | Szostek | August 1969 |
| 3,620,411 | Rump | November 1971 |
| 3,645,384 | Wind | February 1972 |
| 3,800,998 | Gask | April 1974 |
| 3,937,389 | Wind | February 1976 |
| 4,058,212 | Wyslotsky | November 1977 |
| 4,202,464 | Mohs et al | August 1980 |
| 4,456,124 | Kay et al. | June 1984 |
| 4,469,226 | Matney | September 1984 |
| 4,499,353 | Shields | February 1985 |
| 4,512,474 | Harding | April 1985 |

### STATEMENT REGARDING FEDERALLY FUNDED RESEARCH. OR DEVELOPMENT

We the inventors are the sole developers of the Cannula Cover, and have received no Federal Funding regarding the development of this invention.

### REFERENCE TO SEQUENCE LISTING; A TABLE, OR A COMPUTER PROGRAM LISTING COMPACT DISC APPENDIX

Not Applicable

### BACKGROUND OF THE INVENTION

One of the inventors, Robert C. Hagberg, is a prescribed O2 user. On innumerable occasions as Robert lived with the reality of O2 tubing, he would place his Nasal Cannula, safely he thought, on a bedside table while he attended to other needs. When he reached back to retrieve the Cannula Set-Up he found the tubing had fallen to the floor, and on inspection found dirt on the prongs. Then again, on a different occasion Robert was holding the tubing next to the nare prongs so as to blow his nose with a tissue with his other hand; only to see his dog lick the prongs before he could react. Both situations caused Robert to consider alternatives. From his nursing background he knew many care givers have a policy requiring nurses to place the exposed nare prongs of the Nasal Cannula in a zip lock plastic bag while out of a patients nose. His observation, of how reality in the fast paced life of a nurse, noted occasions when policy was not enacted, due mostly to the plastic bag being misplaced, or soiled from falling to the floor itself. Then Roberts' training caused him to be acutely aware of the life cycle of many bacteria, viruses, pathogens, and antibiotic resistant super viruses, some documented by clinical scientific research to be able to live on hard surfaces for up to 21 days.

Once Robert clearly saw and understood the problem, he began thinking of how many other people have had similar situations happen to them. Simultaneously, Robert made himself even more aware of the percentages of nosocomial infections patients suffer while confined to a hospital environment. In conclusion, Robert saw a problem much larger than his personal experience and began thinking of a solution.

Robert approached the second inventor Bruce A. Wachter with the idea of having a clam shell type box attached to the tubing made to fit the actual prongs themselves. Bruce built the box according to Roberts direction, but in the process of shopping for materials to build the box he saw clear 80 Mil PVA tubing on the hardware store shelf and thought that it would make an alternative temporary covering for the Prongs. The second prototype, a 3″×¾″ tubing without protective ends was closer to the answer Robert was looking for but still fell short his goal of protection. Two problems with the second prototype, first, it was not soft and flexible enough and would become a source of irritation on the patients neck while wearing the apparatus; two, the ends were open, thus not separating the briefly unused prongs from everything Robert was attempting to isolate the prongs from.

Robert suggested finding a softer base material to work with and attaching ends with three way valves similar to a hearts valve. Bruce returned to his work shed, and built two new prototypes, one with a three way valve as Robert suggested, and the second with a single slit and two holes the same diameter as the tubes servicing the Nasal Prongs, one hole at either end of the slit. the last prototype met Roberts' needs, and the Cannula Cover was invented. Finally the ends were reduced to only one hole to house the hose and a slit to allow passage of the Nasal Cannula bridge and prongs.

### BRIEF SUMMARY OF THE INVENTION

The Cannula Cover is designed to give immediate, logical, simple, and extended protection for the prongs of an O2 Cannula, isolating the prongs from exposure to pathogens, bacteria, viruses, and antibiotic resistant super viruses, once a Nasal Cannula is removed from a patients nares in the course of daily and medical activity. It is designed to be placed over the tubing of current manufactured Cannula Sets, or it can be attached as an after market add-on, described in subsequent sections of this document. As an added benefit, a shortened hospital stay will result, due to the avoidance of infections not related to the patients original admission, thus providing a financial benefit to the patient, their families, as well as the medical and insurance communities, consequently this invention will lessen the pain and suffering related to nosocomial infections.

### BRIEF DESCRIPTION OF THE SEVERAL VIEWS OF THE DRAWING

FIG. 1 is a x-ray view of the cannula as it sets over both the tubing and the nasal tips, this view shows the holes and slits on opposing ends of the cover.

FIG. 2 Shows and exploded view of the tube made from flat stock and how it is rolled and cemented making it a smooth surface.

FIG. 3 Illustrated how the ends are attached to the flat stock.

FIG. 4 Pictures how the ends will be adhered to the extruded tubing

FIG. 5 Demonstrates how the Cannula Cover is placed on the existing O2 set ups.

FIG. 6 Is a view of the clam shell cover designed as an after market addition.

### DETAILED DESCRIPTION OF THE INVENTION

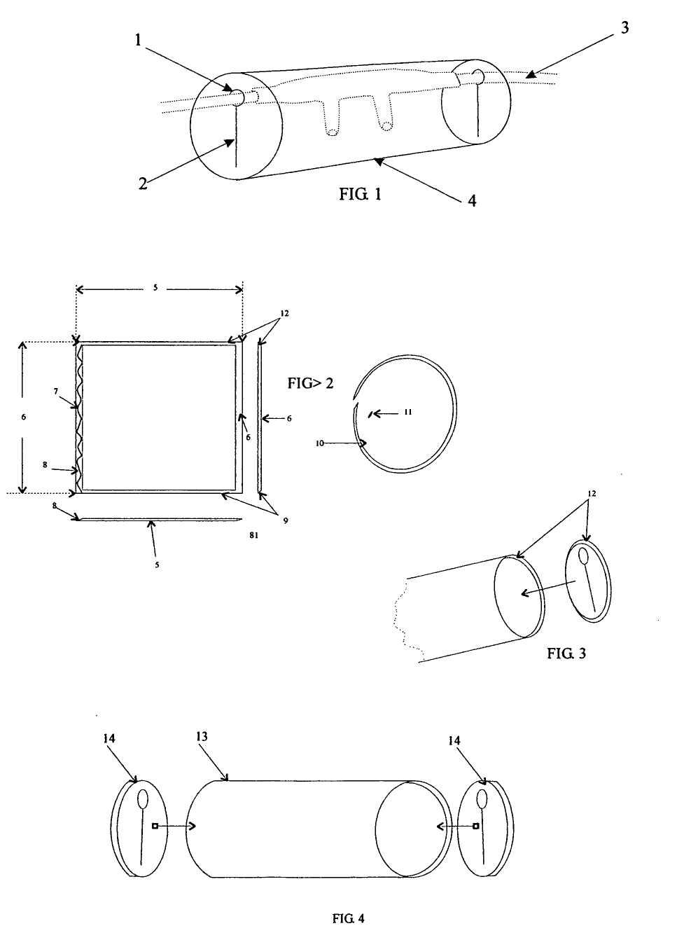

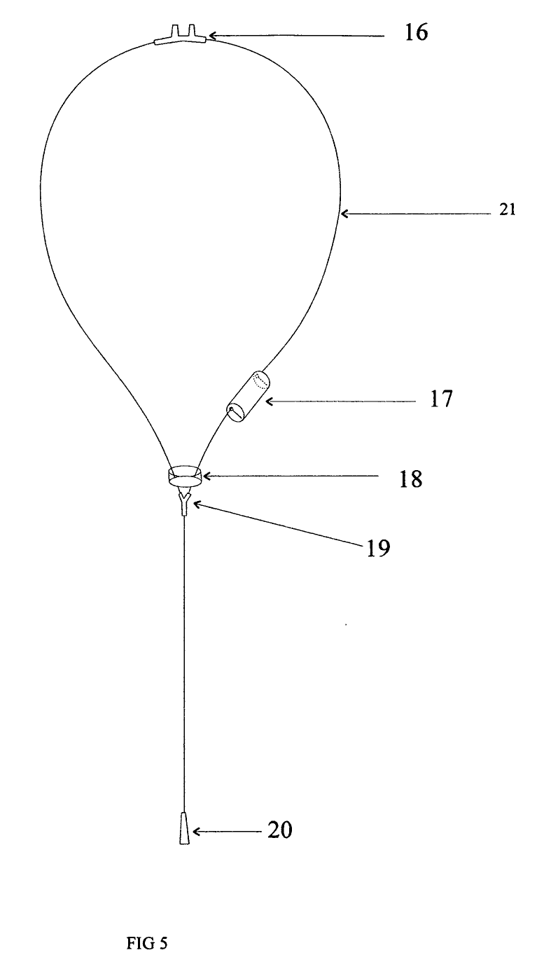

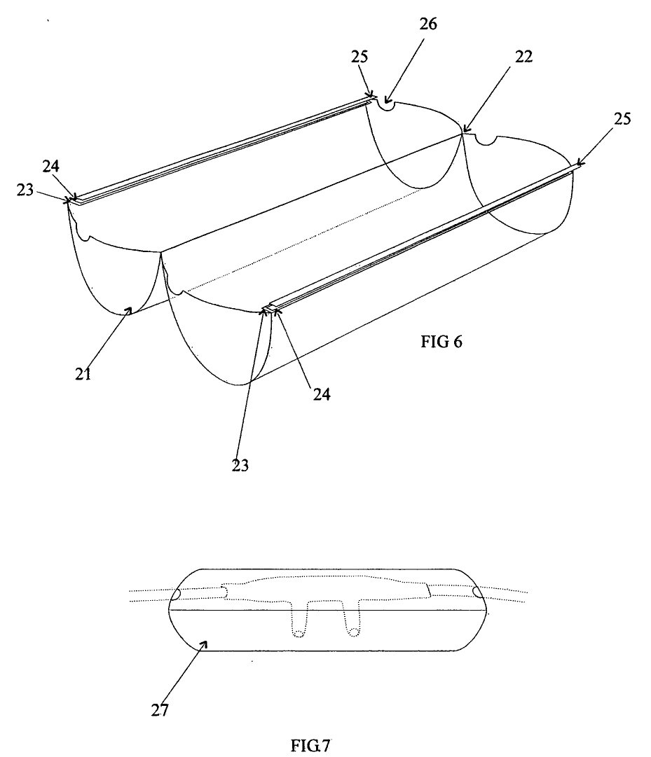

The innovative Cannula Cover is a sterile encasement made to fit on the tubing of current various sized nasal cannulas found on the market shelves. It is designed to be comfortable to the O2 patient, voiding prolonged rubbing of the skin, as allowed in FIG. 5 #17 the Cannula Cover can be slid up and down the subsidiary oxygen supply tubing (FIG. 5 #21), allowing it to not be kept in any one position, which could cause irritation, inflamation and or ulceration. FIG. 1 #'s 1-3 show the Cannula Cover will be measured to fit each different manufactures product (FIG. 1 #3: U.S. Pat. No. 4,106,505, Salter Aug. 15, 1978, being represented as an example) with close tolerances allowing the encasement (FIG. 1 #4) to slide over the subsidiary oxygen supply tubing (FIG. 5 #21) connecting the opposing ends of the nasal cannula. The Cannula Cover will be manufactured in a controlled sanitary environment. The Cannula Cover is designed to be manufactured from either flat stock medical grade plastic (FIGS. 2&3 #'s 5-12), extruded medical grade polyvinyl chloride tubing along with flat stock ends (FIG. 4 #'s 13-14), or should the current Nasal Cannula manufactures choose not to employ this invention on their products as an integral part of their sets we conceive the Cannula Cover to be manufactured as an after market add-on. In the case of using it as an after market product, please refer to (FIG. 6 #′) it will be injection molded of soft flexible polyvinyl chloride type material in a clam shell style design with the longitudinal axis sides (#23) opposing the molded hinge (#22) having a sealable surface (#23) protected by a removable waxed paper strips (#25) allowing the enclosure to be folded over the subsidiary oxygen supply tubes and adhering the opening to itself creating a protective encasement (#27). The after market concept when folded upon itself and the adhesive surfaces engaged will make the Cannula Cover disposable at the end of the current practice use of the Nasal Cannula, insuring a fresh sanitary field for each new Nasal Cannula put into service. The latter two methods of construction are considered to be the most cost effective modes of production. To our knowledge there is no other invention specifically conceived to offer the unique protective function of this invention. The Cannula Covers' usefulness lays in its being designed to slide on and off the Nasal Cannula prongs from either side, due to the holes and slits made on the either end (refer to FIGS. 1,3,4,5 and 6), and its ability to completely enclose the prongs (refer FIGS. 1 &6), thus isolating the prongs from the environment offering a sanitary Nasal set up to be reinstalled into the nares of a patient after brief periods of disengagement.

1.) We claim our inventions' new and useful function is as follows, desiring to gain the security of a patent from the USPTO. Our invention is a light weight, unobtrusively comfortable, sliding, sanitary protective barrier imagined ordinally and specifically to isolate the prongs of O2 Nasal Cannulas from unsanitary fields of contact’, which could allow disease to infiltrate a patients respiratory system via the prongs of the cannula when removed from the nares of a patient and set down haphazardly or otherwise. It is envisioned to be a crush-able shield which will revert back to it's original shape once pressure is released from its surface, allowing the patient to lay on the Cannula Cover without undue discomfort. The same protective thought is also conceptualized to be an aftermarket clam shell style addition to the O2 Nasal Cannula's previously produced as illustrated in FIG. 7.
